(19) United States
(12) Patent Application Publication
(10) Pub. No.: US 2026/0243640 A1
(43) Pub. Date: Aug. 20, 2026

(54) IN-SITU FROZEN SOIL VANE SHEAR APPARATUS

(71) Applicant: AnHui Jianzhu University, Hefei City (CN)

(72) Inventors: Kun HUANG, Hefei City (CN); Guojun CAI, Hefei City (CN); Shuangjie WANG, Xi'an City (CN); Yangcheng CUI, Hefei City (CN); Yi CAI, Hefei City (CN); Ning ZHANG, Hefei City (CN); Dun WU, Hefei City (CN); Kai HUANG, Hefei City (CN); Guangqing HU, Hefei City (CN)

(21) Appl. No.: 19/531,011

(22) Filed: Feb. 5, 2026

(30) Foreign Application Priority Data
Feb. 17, 2025 (CN) 202510169274.1

(51) Int. Cl. G01N 3/24; G01N 33/24

(52) U.S. Cl. CPC G01N 3/24; G01N 33/24

(57) ABSTRACT

An in-situ frozen soil vane shear apparatus is provided, including: a support frame provided with a walking assembly; a locking mechanism, including several locking assemblies arranged on the support frame; a lifting mechanism, including a lifting frame arranged on the support frame, where the lifting frame is movably connected with a lifting assembly for controlling shear tests on frozen soil at different depths; and a shear mechanism, including a shear motor arranged on the lifting assembly, where an output end of the shear motor is in transmission connection with a telescopically arranged shear assembly, and the shear assembly is internally provided with a circulation assembly for controlling temperature in a circulating manner.

### CROSS-REFERENCE TO RELATED APPLICATIONS

This application claims priority to Chinese Patent Application No. 202510169274.1, filed on February 17, 2025, the contents of which are hereby incorporated by reference.

### TECHNICAL FIELD

The present disclosure belongs to the technical field of frozen soil testing, and in particular relates to an in-situ frozen soil vane shear apparatus.

### BACKGROUND

A vane shear apparatus is an indoor geotechnical test instrument used to measure the undrained strength of soft soil or sensitive clay. It may measure the undrained strength of undisturbed soil taken at different depths, and may also conduct tests under different consolidation pressures, as well as determine the sensitivity of soil.

Under low temperature conditions, water inside the soil freezes into ice. Under the action of ice, the strength of the soil increases significantly, causing conventional vane shear apparatuses to withstand greater pressure and be more prone to damage. Meanwhile, the temperature of the frozen soil layer is not constant, but gradually increases towards the side away from the ground, and its performance also changes. However, when the vane moves from a low-temperature position to a high-temperature position, the temperature difference between the vane and the soil causes significant errors between the measured data results and the actual frozen soil parameters, failing to provide data support for studying the geological environment of frozen soil regions.

Therefore, the present disclosure designs an in-situ frozen soil vane shear apparatus to solve the above technical problems.

### SUMMARY

To solve the above technical problems, the present disclosure proposes an in-situ frozen soil vane shear apparatus.

To achieve the above objective, the present disclosure provides an in-situ frozen soil vane shear apparatus, including:

a support frame, where the support frame is provided with a walking assembly for facilitating movement;

a locking mechanism, including several locking assemblies arranged on the support frame, where the locking assemblies extend out of the support frame and are configured for adjusting balance of the support frame, and the locking assemblies are configured for locking the support frame onto a frozen soil ground;

a lifting mechanism, including a lifting frame arranged on the support frame, where the lifting frame is movably connected with a lifting assembly liftably arranged for controlling shear tests on frozen soil at different depths; and

a shear mechanism, including a shear motor arranged on the lifting assembly, where an output end of the shear motor is in transmission connection with a shear assembly telescopically arranged, and the shear assembly is internally provided with a circulation assembly for controlling temperature in a circulating manner.

In some embodiments, the shear assembly includes an end rod in transmission connection with the shear motor, a bottom end of the end rod is connected with several extension rods in an end-to-end sequence, a bottom end of the extension rods is provided with a shear head, the shear head is configured to extend into a frozen soil layer and perform shear tests, and the circulation assembly is arranged within the shear head in a circulating manner.

In some embodiments, the circulation assembly includes a first channel and a second channel opened in the end rod, the first channel is in communication with a first circulation channel within the shear head via a first extension channel arranged within the extension rods, the second channel is in communication with a second circulation channel within the shear head via a second extension channel arranged within the extension rods, and the first circulation channel is in communication with the second circulation channel.

In some embodiments, the lifting assembly is provided with a first circulation tank and a second circulation tank separately arranged, the end rod penetrates through and is rotatably connected to the first circulation tank and the second circulation tank; and the first circulation tank is in communication with the first channel through first communication holes, and the second circulation tank is in communication with the second channel through second communication holes.

In some embodiments, the shear head is provided with two shear plates arranged in a cross shape, the shear plates are internally provided with circulation cavities filling the shear plates, two ends of each of the circulation cavities are respectively in communication with the first circulation channel and the second circulation channel through a third communication hole and a fourth communication hole.

In some embodiments, each of the locking assemblies includes a balance cavity opened on the support frame, a balance rod is liftably arranged in the balance cavity, a bottom end of the balance rod is configured to extend out of a bottom end of the support frame and contact a ground through a balance plate.

In some embodiments, an inner cavity of the balance rod is opened with a locking cavity, a lock rod is liftably arranged in the locking cavity, a bottom end of the lock rod is configured to extend out of the balance plate and pierce into the ground; and the balance rod is threadedly connected with a locking sleeve, a bottom end of the locking sleeve is rotatably connected with a lifting cylinder, and the lifting cylinder is in transmission connection with a top end of the lock rod.

In some embodiments, an outer wall of the balance rod is opened with several first guide grooves, the support frame is provided with first guide blocks adapted to the first guide grooves, and the first guide blocks respectively extend into the first guide grooves and are slidably arranged with the first guide grooves.

In some embodiments, several second guide grooves are penetratingly opened on a side wall of the locking cavity, a side wall of the top end of the lock rod is provided with several second guide blocks adapted to the second guide grooves, the second guide blocks are respectively slidably connected with the second guide grooves; and the second guide blocks extend out of the second guide grooves and are fixedly connected to the lifting cylinder.

In some embodiments, the lifting assembly includes a lifting motor fixedly installed on the lifting frame, an output end of the lifting motor faces downward and is in transmission connection with a lifting screw, a lifting block is threadedly connected to the lifting screw, the lifting block is slidably connected to the lifting frame; and the shear motor, the first circulation tank, and the second circulation tank are respectively fixedly installed on the lifting block.

Compared with the prior art, the present disclosure has the following advantages and technical effects. The present disclosure discloses an in-situ frozen soil vane shear apparatus. The support frame adopts a frame structure, which may reduce the weight of the apparatus, facilitate transportation and transfer, and facilitate field use. The walking assembly is arranged on the support frame, facilitating free movement. The several locking assemblies of the locking mechanism are arranged on the support frame at equal intervals, which may adjust the balance of the support frame during use, facilitating use on uneven ground. After leveling, the locking assemblies may lock the support frame onto the ground, ensuring the balance of the apparatus during shear tests on frozen soil and avoiding test errors caused by sliding due to shear reaction forces. The lifting frame of the lifting mechanism is installed on the support frame, and the shear motor of the shear mechanism is installed on the lifting assembly. The lifting assembly drives the shear motor to lift, thereby driving the shear assembly to lift, so that the bottom end of the shear assembly extends into the frozen soil for shear tests. Meanwhile, the length of the shear assembly may be easily adjusted, thereby conveniently adjusting the depth to which the shear assembly extends into the frozen soil, improving the applicability of the apparatus. The circulation assembly may, according to the frozen soil temperature detected by the shear assembly, regulate the temperature of the shear assembly through the circulating circulation fluid, so that the temperature of the shear assembly matches the external ambient temperature, thereby facilitating the conduct of shear tests, avoiding shear test errors caused by the temperature difference between the shear assembly and the frozen soil, improving the authenticity of data obtained from shear tests, and providing more detailed data for the study of frozen soil characteristics.

The present disclosure is simple in structure, and convenient to use. It can be easily transported to field frozen soil areas for shear tests, and may conveniently adjust the balance of the apparatus and lock the apparatus. The setting with lifting adjustment function allows for shear tests on frozen soil at different depths, and ensures that the temperature of the shear area matches the external environment, improving the accuracy of shear test data.

### BRIEF DESCRIPTION OF THE DRAWINGS

The drawings forming a part of the present disclosure are used to provide a further understanding of the present disclosure. The schematic embodiments of the present disclosure and their descriptions are used to explain the present disclosure and do not constitute an improper limitation of the present disclosure. In the drawings:

FIG. 1 is an axonometric view of an in-situ frozen soil vane shear apparatus according to Embodiment 1 of the present disclosure.

FIG. 2 is a side view of the in-situ frozen soil vane shear apparatus according to the present disclosure.

FIG. 3 is a schematic diagram of a locking mechanism according to the present disclosure.

FIG. 4 is a partially enlarged cross-sectional view of portion A in FIG. 3 of the present disclosure.

FIG. 5 is an axonometric view of a balance rod according to the present disclosure.

FIG. 6 is a structural schematic diagram of an end rod according to the present disclosure.

FIG. 7 is a structural schematic diagram of a shear plate according to the present disclosure.

FIG. 8 is a structural diagram of a detection mechanism of Embodiment 2 of the present disclosure.

FIG. 9 is a partially enlarged view of portion B in FIG. 8 of the present disclosure.

FIG. 10 is a partial enlarged view of portion C in FIG. 8 of the present disclosure.

FIG. 11 is a schematic diagram of a first connection assembly of Embodiment 3 of the present disclosure.

FIG. 12 is a schematic diagram of a second connection assembly of Embodiment 3 of the present disclosure.

### DETAILED DESCRIPTION OFTHE EMBODIMENTS

In the following, the technical solutions in the embodiments of the present disclosure will be clearly and completely described with reference to the attached drawings. Apparently, the described embodiments are only a part of the embodiments of the present disclosure, but not all the embodiments. Based on the embodiments in the present disclosure, all other embodiments obtained by one of ordinary skill in the art without creative effort belong to the protection scope of the present disclosure.

In order to make the above objects, features and advantages of the present disclosure more clear and easier to understand, the present disclosure will be further described in detail with the attached drawings and specific embodiments.

### Embodiment 1

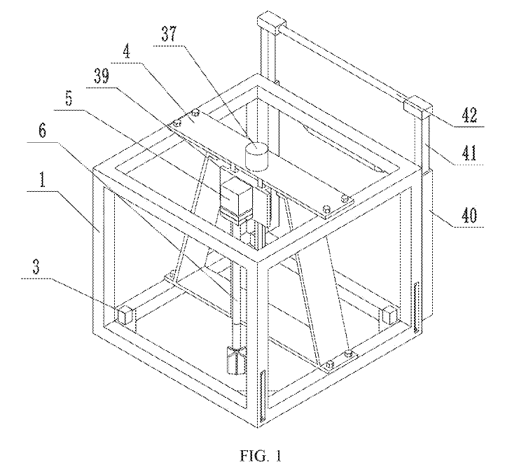

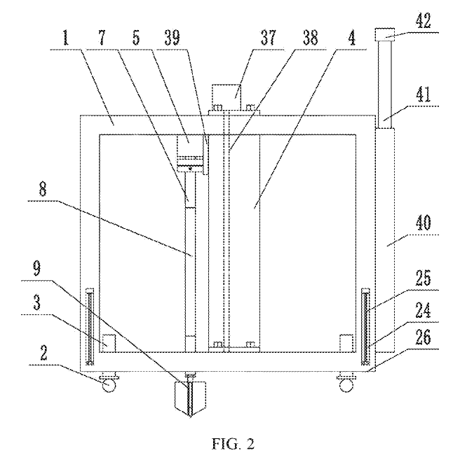

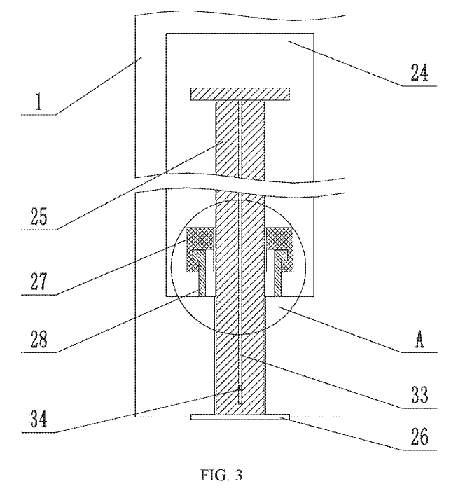

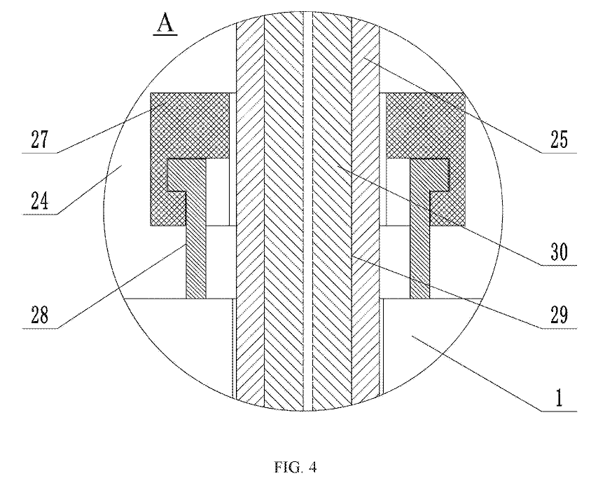

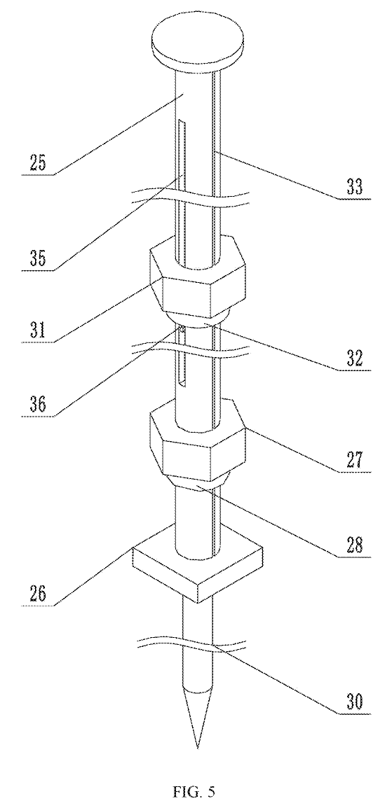

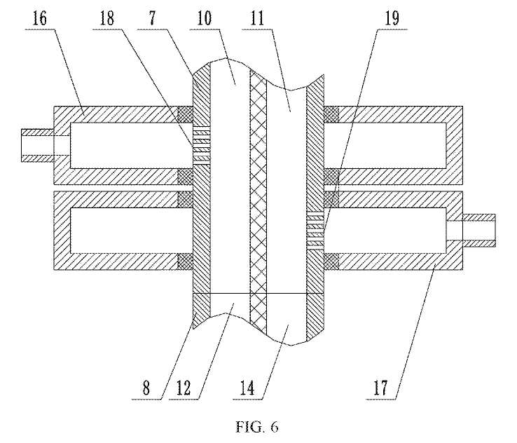

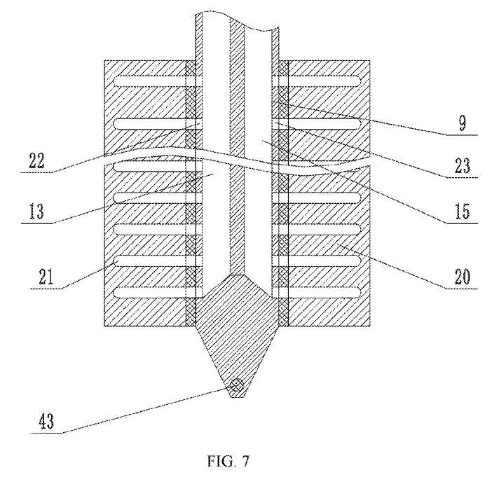

With reference to FIG. 1 to FIG. 7, this embodiment provides an in-situ frozen soil vane shear apparatus, including: a support frame 1, a locking mechanism, a lifting mechanism, and a shear mechanism.

The support frame 1 is provided with a walking assembly for facilitating movement.

The locking mechanism includes several locking assemblies arranged on the support frame 1, the locking assemblies extend out of the support frame 1 and are used for adjusting the balance of the support frame 1, and the locking assemblies are used for locking the support frame 1 onto frozen soil ground.

The lifting mechanism includes a lifting frame 4 arranged on the support frame 1, where the lifting frame 4 is movably connected with a lifting assembly for controlling shear tests on frozen soil at different depths, and the lifting assembly is configured for being lifted and lowered.

The shear mechanism includes a shear motor 5 arranged on the lifting assembly, where the output end of the shear motor 5 is in transmission connection with a telescopic shear assembly 6, the shear assembly 6 is internally provided with a circulation assembly for controlling temperature in a circulating manner.

The present disclosure discloses the in-situ frozen soil vane shear apparatus. The support frame 1 is made of a frame structure, which may reduce the weight of the apparatus, facilitate transportation and transfer, and facilitate field use. The walking assembly is arranged on the support frame 1, facilitating free movement. The several locking assemblies of the locking mechanism are arranged on the support frame 1 at equal intervals, which may adjust the balance of the support frame 1 during use, facilitating use on uneven ground. After leveling, the locking assemblies may lock the support frame 1 onto the ground, ensuring the balance of the apparatus during shear tests on frozen soil and avoiding test errors caused by sliding due to shear reaction forces. The lifting frame 4 of the lifting mechanism is installed on the support frame 1, and the shear motor 5 of the shear mechanism is installed on the lifting assembly. The lifting assembly drives the shear motor 5 to lift, thereby driving the shear assembly 6 to lift, so that the bottom end of the shear assembly 6 extends into the frozen soil for shear tests. Meanwhile, the length of the shear assembly 6 may be easily adjusted, thereby conveniently adjusting the depth to which the shear assembly 6 extends into the frozen soil, improving the applicability of the apparatus. The circulation assembly may, according to the frozen soil temperature detected by the shear assembly 6, regulate the temperature of the shear assembly 6 through the circulating circulation fluid, so that the temperature of the shear assembly 6 matches the external ambient temperature, thereby facilitating the conduct of shear tests, avoiding shear test errors caused by the temperature difference between the shear assembly 6 and the frozen soil, improving the authenticity of data obtained from shear tests. The present disclosure has a simple structure, is convenient to use, may be easily transported to field frozen soil areas for shear tests, and may conveniently adjust the balance of the apparatus and lock the apparatus. The setting with the lifting adjustment function allows for shear tests on frozen soil at different depths, and ensures that the temperature of the shear area matches the external environment, improving the accuracy of shear test data.

In the embodiment, the support frame 1 adopts a frame-type structure, ensuring the lightweight nature of the apparatus itself and allowing flexible adjustment of the installation positions of internal devices.

In the embodiment, to improve the protective performance of the apparatus, protective plates may be attached to the outside of the support frame 1 to prevent external dust and impurities from affecting the apparatus, enhancing the protective performance of the apparatus.

In the embodiment, the walking assembly of this embodiment includes walking frames 3 arranged on the support frame 1, the bottom ends of the walking frames 3 extend out of the support frame 1 and are respectively provided with walking wheels 2, facilitating the movement of the support frame 1.

In the embodiment, one side of the support frame 1 is provided with connection cylinders 40, a telescopic rod 41 is liftably arranged within each of the connection cylinders 40, a handle 42 is arranged between two telescopic rods 41, facilitating pushing or pulling the apparatus. Meanwhile, the height of the handle 42 may be adjusted to facilitate different users to control the support frame 1.

In the embodiment, the shear assembly 6 includes an end rod 7 in transmission connection with the shear motor 5, the bottom end of the end rod 7 is connected with several extension rods 8, the extension rods 8 are arranged in an end-to-end sequence. The bottom end of the extension rods 8 is provided with a shear head 9, the shear head 9 may extend into the frozen soil layer and perform shear tests, the circulation assembly is arranged within the shear head 9 in a circulating manner. The end rod 7 is in transmission connection with the output end of the shear motor 5, providing the required shear force for shear tests; the bottom end of the end rod 7 may be end-to-end connected to several sections of extension rods 8, allowing adjustment of the overall length of the extension assembly, thereby controlling the depth at which the shear head 9 penetrates into the frozen soil. After use, the extension rods 8 may be easily disassembled, facilitating transportation and storage; the circulation fluid of the circulation assembly passes through the end rod 7 and the several sections of extension rods 8 and then circulates within the shear head 9, adjusting the temperature of the shear head 9.

In the embodiment, a shear force sensor is arranged between the end rod 7 and the shear motor 5 of this embodiment to measure the shear force received by the shear head 9 when shearing frozen soil.

In the embodiment, a temperature sensor 43 is arranged inside the shear head 9 of this embodiment, the temperature sensor 43 is used to measure the temperature at the shear position, the temperature data is transmitted to the external circulation devices, thereby allowing control of the temperature of the circulating circulation fluid to adjust the temperature of the shear head 9.

In the embodiment, the circulation assembly includes a first channel 10 and a second channel 11 opened in the end rod 7, the first channel 10 is in communication with a first circulation channel 13 within the shear head 9 via a first extension channel 12 arranged within the extension rods 8, the second channel 11 is in communication with a second circulation channel 15 within the shear head 9 via a second extension channel 14 arranged within the extension rods 8, the first circulation channel 13 is in communication with the second circulation channel 15. The first channel 10 and second channel 11 in the end rod 7 are used for the inlet and outlet circulation of the circulation fluid. The circulation fluid enters the first circulation channel 13 within the shear head 9 from the first channel 10 through the first extension channel 12, then passes from the second circulation channel 15 within the shear head 9 through the second extension channel 14 into the second channel 11, enabling the circulation fluid to flow in a circulating manner, facilitating control of the flow of the circulation fluid and thereby adjusting the temperature of the shear head 9.

In the embodiment, the lifting assembly is provided with a first circulation tank 16 and a second circulation tank 17, the first circulation tank 16 and the second circulation tank 17 are separately arranged, the end rod 7 penetrates through the first circulation tank 16 and the second circulation tank 17 and is rotatably connected to the first circulation tank 16 and the second circulation tank 17. The first circulation tank 16 is in communication with the first channel 10 through first communication holes 18, the second circulation tank 17 is in communication with the second channel 11 through second communication holes 19. The first circulation tank 16 and the second circulation tank 17 are respectively connected to external circulation devices, one for the intake of the circulation fluid, the other for the discharge of the circulation fluid. Meanwhile, the end rod 7 is rotatably arranged with the first circulation tank 16 and the second circulation tank 17 respectively through sealing bearings, ensuring the rotation of the end rod 7 without affecting the connections of the first circulation tank 16 and the second circulation tank 17.

In the embodiment, the shear head 9 is provided with two shear plates 20 arranged in a cross shape, the shear plates 20 are internally provided with circulation cavities 21 filling the shear plates 20, two ends of each of the circulation cavities 21 are respectively in communication with the first circulation channel 13 and the second circulation channel 15 through a third communication hole 22 and a fourth communication hole 23. The circulation cavities 21 are arranged within the shear plates 20, the two ends of each of the circulation cavities 21 are respectively in communication with the first circulation channel 13 and the second circulation channel 15, thereby allowing the circulation fluid to flow through the circulation cavities 21, and adjusting the temperature of the shear plates 20 through the temperature of the circulation fluid within the circulation cavities 21.

In the embodiment, the first communication holes 18 and the second communication holes 19 of this embodiment are set as multiple holes to facilitate the flow of the circulation fluid.

In the embodiment, each of the locking assemblies includes a balance cavity 24 opened on the support frame 1, where the balance cavity 24 is provided with a balance rod 25, the balance rod 25 is configured for being lifted and lowered, the bottom end of the balance rod 25 extends out of the bottom end of the support frame 1 and contacts the ground through a balance plate 26. The bottom end of the balance cavity 24 is rotatably connected with a balance sleeve 27, the balance sleeve 27 is threadedly connected with the outer wall of the balance rod 25. The outer wall of the balance rod 25 is opened with several first guide grooves 33, the support frame 1 is provided with first guide blocks 34 adapted to the first guide grooves 33, and the first guide blocks 34 extend into the first guide grooves 33 and are slidably arranged with the first guide grooves 33. The bottom end of the balance cavity 24 is opened with a through hole, the balance rod 25 passes through the through hole and is provided with the balance plate 26, the first guide blocks 34 are arranged within the through hole, ensuring that the balance rod 25 only performs up-and-down motion without rotation. The bottom end of the balance cavity 24 is fixedly connected with a balance tube 28, the balance sleeve 27 is rotatably connected on the balance tube 28. When the balance sleeve 27 rotates, the lifting of the balance rod 25 may be controlled through the threaded connection between the balance sleeve 27 and the balance rod 25, thereby controlling the extension length of the balance plate 26, adjusting the balance level of the support frame 1. Simultaneously, during testing, the balance rod 25 lifts the walking wheel 2 at the bottom of the support frame 1 off the ground, preventing the walking wheel from wobbling.

In the embodiment, the inner cavity of the balance rod 25 is opened with a locking cavity 29, the locking cavity 29 is provided with a lock rod 30, the lock rod 30 is configured for being lifted, the bottom end of the lock rod 30 extends out of the balance plate 26 and pierces into the ground. The balance rod 25 is threadedly connected with a locking sleeve 31, the bottom end of the locking sleeve 31 is rotatably connected with a lifting cylinder 32, the lifting cylinder 32 is in transmission connection with the top end of the lock rod 30. The side wall of the locking cavity 29 is opened with several second guide grooves 35, and the second guide grooves 35 penetrate the side wall of the locking cavity. The side wall of the top end of the lock rod 30 is provided with several second guide blocks 36 adapted to the second guide grooves 35, the second guide blocks 36 are respectively slidably connected with the second guide grooves 35. The second guide blocks 36 extend out of the second guide grooves 35 and are fixedly connected to the lifting cylinder 32. The lock rod 30 is slidably connected within the locking cavity 29, the second guide blocks 36 at the top end of the lock rod 30 extend out of the second guide grooves 35 and are fixedly connected to the lifting cylinder 32. When the locking sleeve 31 rotates, through the threaded connection between the locking sleeve 31 and the balance rod 25, the lifting cylinder 32 is driven to lift and lower, thereby driving the lock rod 30 to lift and lower, causing the pointed tip at the bottom end of the lock rod 30 to extend out of the balance plate 26 and pierce into the frozen soil, thereby ensuring stability during shearing.

In the embodiment, the lifting assembly includes a lifting motor 37 fixedly installed on the lifting frame 4, the output end of the lifting motor 37 faces downward and is in transmission connection with a lifting screw 38, a lifting block 39 is threadedly connected to the lifting screw 38, the lifting block 39 is slidably connected with the lifting frame 4. The shear motor 5, the first circulation tank 16 and the second circulation tank 17 are respectively fixedly installed on the lifting block 39. The lifting motor 37 drives the lifting block 39 to lift through the lifting screw 38, thereby controlling the lifting of the shear motor 5, providing power for piercing into the frozen soil, and combining with the downward drilling of the shear plates 20 during shearing.

### Embodiment 2

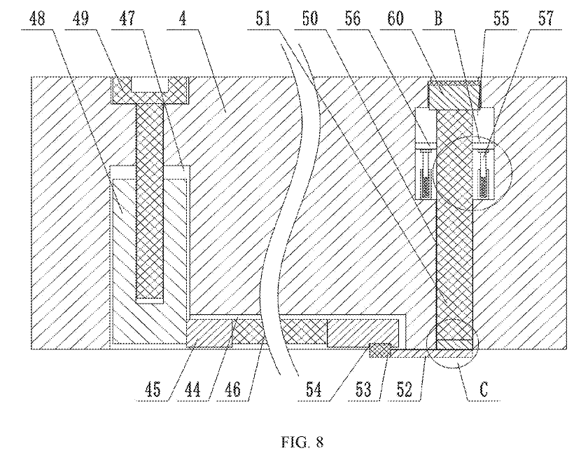

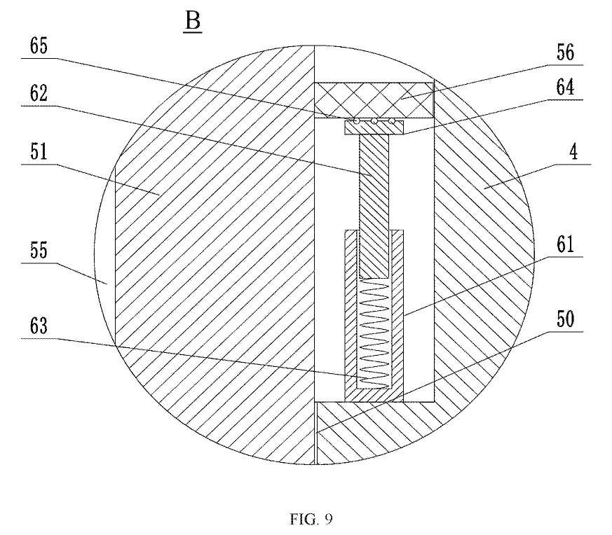

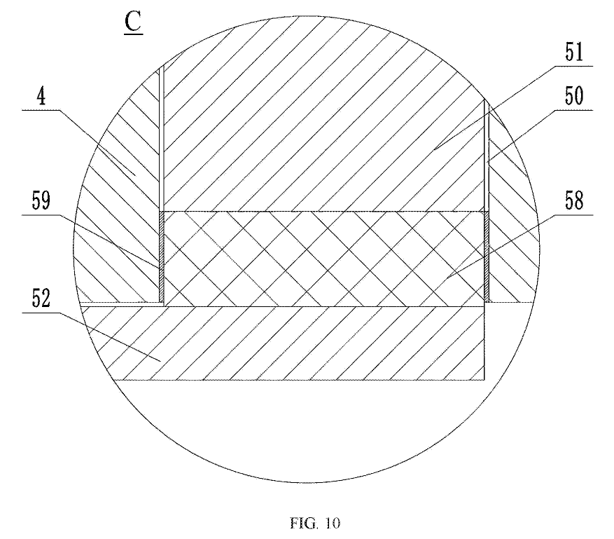

With reference to FIG. 8 to FIG. 10, the difference between this embodiment and Embodiment 1 is that the lifting frame 4 of this embodiment is provided with a detection mechanism for detecting the geological environment below, used for detecting the target area before drilling tests, to discover stones or other objects that may affect drilling within the detection area in advance, ensuring smooth drilling progress, avoiding damage to the shear head 9, and guaranteeing the progress of the test.

The detection mechanism of this embodiment includes a detection assembly movably arranged at the bottom end of the lifting frame 4, and an adjustment assembly for controlling the movement and fixation of the detection assembly is arranged inside the lifting frame 4. Before use, the detection assembly is unlocked by the adjustment assembly, then the detection assembly is adjusted to a position corresponding to the shear head 9 to detect the area below the target, ensuring there are no stones or other debris in the lower area. If stones or other debris that affect drilling tests are found, the detection position needs to be changed or the affecting debris needs to be removed in advance. After use, the detection assembly is retracted and fixed via the adjustment assembly, and then drilling tests are conducted.

The detection assembly includes a detection groove 44 opened below the lifting frame 4, a detection frame 45 is detachably connected within the detection groove 44, the detection frame 45 may move into and out of the detection groove 44 driven by the adjustment assembly, facilitating storage and use; the detection frame 45 is provided with a detection module 46 for detection. When the detection frame 45 is not in use, the adjustment assembly locks the detection frame 45 within the detection groove 44 so the detection frame 45 does not protrude, avoiding collisions and improving its protective performance. During the usage of the detection frame 45, the detection frame 45 is pushed out of the detection groove 44 through the adjustment assembly, then rotated into position so that the detection module 46 moves to a position corresponding to the shear head 9, and then detects stones and other debris underground. After detection is completed, subsequent processing is performed based on the detection results. After use, the detection frame 45 is retracted back into the detection groove 44 through the adjustment assembly and locked in place, facilitating subsequent reuse.

In the embodiment, the detection module 46 of this embodiment is a conventional geological detection device, which may use media including but not limited to electromagnetic, vibration, gravity, or ultrasonic waves for detection. The principle and usage of the detection module 46 may refer to the KDZ1114-8C30 Mine Geological Detector or the DTC200 Explosion-Proof Geological Advance Detector, and will not be detailed here.

The adjustment assembly includes an adjustment module for controlling the lifting and rotation of the detection frame 45 and a locking module for supporting and locking the detection frame 45. The locking module supports and locks the free end of the detection frame 45 when the detection frame 45 is retracted into the detection groove 44. During use, the locking module is rotated away to allow the detection frame 45 to freely move into and out of the detection groove 44. After use, the detection frame 45 is retracted back into the detection groove 44 by the adjustment module and supported and locked by the locking module.

The adjustment module includes an adjustment groove 47 opened on the lifting frame 4, an adjustment block 48 is slidably arranged within the adjustment groove 47 for vertical movement, the top end of the adjustment block 48 is threadedly connected with an adjustment bolt 49, the adjustment bolt 49 penetrates through the bottom plate of the lifting frame 4 and is rotatably connected with the lifting frame 4, the bottom end side wall of the adjustment groove 47 is in communication with the detection groove 44, the end of the detection frame 45 is fixedly connected to the side wall of the adjustment block 48, the detection frame 45 is driven to lift through the lifting of the adjustment block 48, thereby controlling the use of the detection frame 45. During use, by rotating the adjustment bolt 49 exposed at the top of the lifting frame 4, the lifting of the adjustment block 48 is controlled, thereby driving the detection frame 45 to lift.

In the embodiment, the side wall of the adjustment block 48 is provided with several limit grooves, the side wall of the adjustment groove 47 is provided with several limit strips respectively adapted to the limit grooves, causing the adjustment block 48 to only perform up-and-down motion without the rotation along with the adjustment bolt 49.

The locking module includes a locking hole 50 penetratingly opened in the lifting frame 4, a locking rod 51 is movably arranged within the locking hole 50. The bottom end of the locking rod 51 extends out of the locking hole 50, and is perpendicularly and fixedly connected to one end of a support plate 52, the top end of the other end of the support plate 52 is provided with a flexible contact block 53. When the detection module is not in use, the free end of the support plate 52 is turned towards the detection frame 45 driven by the locking rod 51, the contact block 53 supports the free end of the detection frame 45, supporting and locking the detection frame 45.

In the embodiment, the bottom end of the detection frame 45 is provided with a contact groove 54 adapted to the contact block 53. When supporting and locking the detection frame 45, the contact block 53 is clamped in the contact groove 54, improving locking stability.

In the embodiment, a middle part of the locking hole 50 is opened with an accommodating groove 55 coaxial with the locking hole 50, the area of the locking rod 51 passing through the accommodating groove 55 is provided with an expansion plate 56, allowing the expansion plate 56 to lift and rotate within the accommodating groove 55. The outer diameter of the expansion plate 56 is larger than the diameter of the locking hole 50, preventing the expansion plate 56 from coming out.

In the embodiment, the bottom end of the accommodating groove 55 is provided with several lifting support rods 57, the top ends of the lifting support rods 57 support the bottom surface of the expansion plate 56. During use, the locking rod 51 is pressed down, causing the locking rod 51 to move downward along the locking hole 50, thereby causing the support plate 52 to lower the contact block 53, separating the contact block 53 from the detection frame 45. Then, the locking rod 51 is rotated, so that the support plate 52 no longer blocks the lifting of the detection frame 45. After rotation, the locking rod 51 is released, and the lifting support rods 57 drive the locking rod 51 to reset by supporting the expansion plate 56, facilitating subsequent locking of the detection frame 45 again.

In the embodiment, the bottom end of the locking rod 51 is provided with a prismatic guide section 58, the bottom end of the locking hole 50 is provided with a locking section 59 adapted to the guide section 58. When not in use, the guide section 58 is pushed up by the lifting support rods 57, the guide section 58 engages with the locking section 59, preventing the locking rod 51 from deflecting, improving locking stability. During use, the top end of the locking rod 51 is pressed down with a tool, causing the guide section 58 to move down and disengage from the locking section 59, then the locking rod 51 may be easily rotated, for example, by a tool, separating the contact block 53 from the detection frame 45.

In the embodiment, the top end of the locking rod 51 of this embodiment is provided with a positioning head 60. A fixed angle limiting module is arranged between the positioning head 60 and the locking hole 50, the set angle is adapted to the number of prisms of the guide section 58 and the locking section 59, allowing the locking rod 51 to deflect at a specific angle.

In the embodiment, each of the lifting support rods 57 includes a bottom cylinder 61 fixed at the bottom end of the accommodating groove 55, a movable rod 62 is liftably arranged within the bottom cylinder 61, a support spring 63 is arranged between the bottom end of the movable rod 62 and the bottom cylinder 61 for supporting the rebound of the movable rod 62. The top end of the movable rod 62 extends out of the bottom cylinder 61 and is fixedly connected with a support block 64, the top end of the support block 64 is in rolling contact with the bottom end of the expansion plate 56 through balls 65, so that when supporting the expansion plate 56, it does not affect the rotation of the locking rod 51.

### Embodiment 3

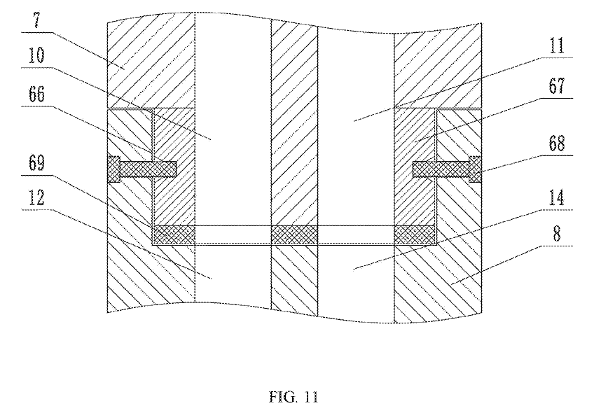

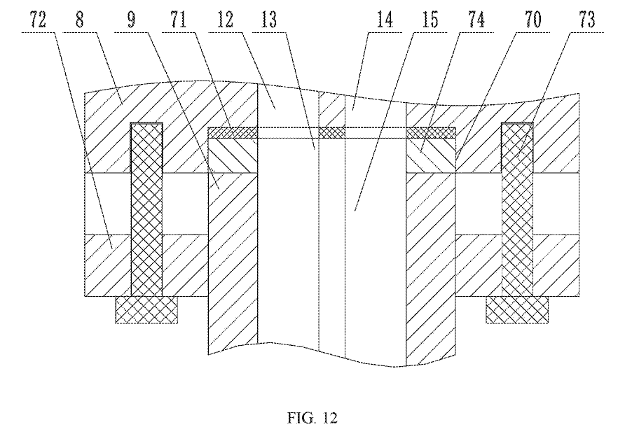

With reference to FIG. 11 to FIG. 12, in this embodiment, the bottom end of the end rod 7 and the extension rod 8 at the top end are connected through a first connection assembly, the bottom end of the bottommost extension rod 8 is connected to the top end of the shear head 9 through a second connection assembly, facilitating the sealed connection of the internal circulation channels and enabling fluid flow.

The first connection assembly includes a connection groove 66 opened at the top end of the extension rod 8 at the top end, the bottom end of the end rod 7 is provided with a connection head 67 adapted to the connection groove 66. Both the connection head 67 and the connection groove 66 are provided with specific prismatic shapes, facilitating the corresponding communication between the first channel 10 and the first extension channel 12. The side wall of the extension rod 8 at the top end is provided with locking through holes, and the side wall of the connection head 67 is opened with locking screw holes adapted to the locking through holes. During connection, the connection head 67 is inserted into the connection groove 66, the locking through holes and the locking screw holes are aligned, and then locked and fixed by first bolts 68, connecting the extension rod 8 at the top end and the end rod 7 together, ensuring connection stability and the normal communication of the internal channels.

In the embodiment, the cross-sections of the connection head 67 and the connection groove 66 are both set to specific shapes, so that when the connection head 67 and the connection groove 66 are connected, the corresponding channels may align precisely, avoiding confusion.

In the embodiment, the inlet of the locking screw hole is provided with a flared opening, facilitating the first bolt 68 from the locking through hole to thread into the locking screw hole.

In the embodiment, a first sealing gasket 69 is arranged between the connection head 67 and the connection groove 66, increasing the sealing of the connection.

In the embodiment, both ends of each of the extension rods 8 of this embodiment are respectively provided with a connection head 67 and a connection groove 66, facilitating the connection of multiple extension rods 8 for testing formations at different depths.

The second connection assembly includes an installation groove 70 opened at the bottom end of the last section of extension rod 8, the top end of the shear head 9 is provided with an installation head 74 adapted to the installation groove 70. During connection, the installation head 74 is inserted into the installation groove 70. The shear head 9 is provided with a connection plate 72 adapted to the cross-section of the last section of extension rod 8, the connection plate 72 is provided with several second bolts 73, the connection plate 72 is pressed against the bottom end of the last section of extension rod 8 through the second bolts 73 to complete fixation.

In the embodiment, both the installation groove 70 and the installation head 74 are set to specific prismatic shapes, aligning the channels between the last section of extension rod 8 and the shear head 9.

In the embodiment, a second sealing gasket 71 is arranged between the installation groove 70 and the installation head 74, causing the channels within the last section of extension rod 8 to align and be in communication with the channels within the shear head 9, with precise positioning and high sealing.

In the description of the present disclosure, it should be understood that the terms “longitudinal”, “transverse”, “up”, “down”, “front”, “back”, “left”, “right”, “vertical”, “horizontal”, “top”, “bottom”, “inside”, “outside”, etc. indicate orientation or positional relationships based on the orientation or positional relationships shown in the accompanying drawings, only for the convenience of describing the present disclosure, and do not indicate or imply that the device or element referred to must have a specific orientation, be constructed and operated in a specific orientation, and therefore cannot be understood as a limitation of the present disclosure.

The above-mentioned embodiments only describe the preferred mode of the present disclosure, and do not limit the scope of the present disclosure. Under the premise of not departing from the design spirit of the present disclosure, various modifications and improvements made by one of ordinary skill in the art to the technical solution of the present disclosure should fall within the protection scope defined by the claims of the present disclosure.

What is claimed is:

1. An in-situ frozen soil vane shear apparatus, comprising:
a support frame, wherein the support frame is provided with a walking assembly for facilitating movement;
a locking mechanism, comprising a plurality of locking assemblies arranged on the support frame, wherein the locking assemblies extend out of the support frame and are configured for adjusting balance of the support frame, and the locking assemblies are configured for locking the support frame onto a frozen soil ground;
a lifting mechanism, comprising a lifting frame arranged on the support frame, wherein the lifting frame is movably connected with a lifting assembly liftably arranged for controlling shear tests on frozen soil at different depths; and
a shear mechanism, comprising a shear motor arranged on the lifting assembly, wherein an output end of the shear motor is in transmission connection with a shear assembly telescopically arranged, and the shear assembly is internally provided with a circulation assembly for controlling temperature in a circulating manner;
wherein the lifting frame is provided with a detection mechanism for detecting a geological environment below, the detection mechanism comprises a detection assembly movably arranged at a bottom end of the lifting frame, and the lifting frame is internally provided with an adjustment assembly for controlling movement and fixation of the detection assembly;
the detection assembly comprises a detection groove opened below the lifting frame, a detection frame is detachably connected within the detection groove, and the detection frame is capable of moving into and out of the detection groove driven by the adjustment assembly;
the adjustment assembly comprises an adjustment module for controlling lifting and rotation of the detection frame and a locking module for supporting and locking the detection frame, the locking module comprises a locking hole penetratingly opened in the lifting frame, a locking rod is movably arranged in the locking hole, a bottom end of the locking rod extends out of the locking hole and is perpendicularly and fixedly connected to one end of a support plate, a top end of another end of the support plate is provided with a flexible contact block, and the contact block is configured for supporting a free end of the detection frame;
a bottom end of the detection frame is provided with a contact groove adapted to the contact block, and when supporting and locking the detection frame, the contact block is clamped in the contact groove; and
the bottom end of the locking rod is provided with a prismatic guide section, a bottom end of the locking hole is provided with a locking section adapted to the guide section, when not in use, the locking section is pushed up and engages with the guide section, and when in use, the locking rod is pressed down, causing the guide section to move down and disengage from the locking section.

2. The in-situ frozen soil vane shear apparatus according to claim 1, wherein the shear assembly comprises an end rod in transmission connection with the shear motor, a bottom end of the end rod is connected with a plurality of extension rods in an end-to-end sequence, a bottom end of the extension rods is provided with a shear head, the shear head is configured to extend into a frozen soil layer and perform the shear tests, and the circulation assembly is arranged within the shear head in the circulating manner.

3. The in-situ frozen soil vane shear apparatus according to claim 2, wherein the circulation assembly comprises a first channel and a second channel opened in the end rod, the first channel is in communication with a first circulation channel within the shear head via a first extension channel arranged within the extension rods, the second channel is in communication with a second circulation channel within the shear head via a second extension channel arranged within the extension rods, and the first circulation channel is in communication with the second circulation channel.

4. The in-situ frozen soil vane shear apparatus according to claim 3, wherein the lifting assembly is provided with a first circulation tank and a second circulation tank separately arranged, the end rod penetrates through and is rotatably connected to the first circulation tank and the second circulation tank, and the first circulation tank is in communication with the first channel through first communication holes, and the second circulation tank is in communication with the second channel through second communication holes.

5. The in-situ frozen soil vane shear apparatus according to claim 3, wherein the shear head is provided with two shear plates arranged in a cross shape, the two shear plates are internally provided with circulation cavities filling the two shear plates, and two ends of each of the circulation cavities are respectively in communication with the first circulation channel and the second circulation channel through a third communication hole and a fourth communication hole.

6. The in-situ frozen soil vane shear apparatus according to claim 1, wherein each of the locking assemblies comprises a balance cavity opened on the support frame, a balance rod is liftably arranged in the balance cavity, and a bottom end of the balance rod is configured to extend out of a bottom end of the support frame and contact the frozen soil ground through a balance plate.

7. The in-situ frozen soil vane shear apparatus according to claim 6, wherein an inner cavity of the balance rod is opened with a locking cavity, a lock rod is liftably arranged in the locking cavity, a bottom end of the lock rod is configured to extend out of the balance plate and pierce into the frozen soil ground, the balance rod is threadedly connected with a locking sleeve, a bottom end of the locking sleeve is rotatably connected with a lifting cylinder, and the lifting cylinder is in transmission connection with a top end of the lock rod.

8. The in-situ frozen soil vane shear apparatus according to claim 6, wherein an outer wall of the balance rod is opened with a plurality of first guide grooves, the support frame is provided with first guide blocks adapted to the first guide grooves, and the first guide blocks respectively extend into the first guide grooves and are slidably arranged with the first guide grooves.

9. The in-situ frozen soil vane shear apparatus according to claim 7, wherein a plurality of second guide grooves are penetratingly opened on a side wall of the locking cavity, a side wall of the top end of the lock rod is provided with a plurality of second guide blocks adapted to the second guide grooves, the second guide blocks are respectively slidably connected with the second guide grooves, and the second guide blocks extend out of the second guide grooves and are fixedly connected to the lifting cylinder.

10. The in-situ frozen soil vane shear apparatus according to claim 4, wherein the lifting assembly comprises a lifting motor fixedly installed on the lifting frame, an output end of the lifting motor faces downward and is in transmission connection with a lifting screw, a lifting block is threadedly connected to the lifting screw, the lifting block is slidably connected to the lifting frame, and the shear motor, the first circulation tank and the second circulation tank are respectively fixedly installed on the lifting block.
